(19) United States
(12) Patent Application Publication
(10) Pub. No.: US 2002/0066756 A1
(43) Pub. Date: Jun. 6, 2002

(54) Multiple spray dispenser

(76) Inventor: Janice Kinsman, Gilbert, AZ (US)

Correspondence Address: RICHARD R. MYBECK, Post Office Box 5540, Scottsdale, AZ, 85261-5540, US

(21) Appl. No.: 09/989,510

(22) Filed: Nov. 20, 2001

Related U.S. Application Data
(60) Provisional application No. 60/252,362, filed on Nov. 21, 2000.

(51) Int. Cl. B67D 3/00

(52) U.S. Cl. 222/547

(57) ABSTRACT

A multiple spray apparatus for portable use is reported. The spray apparatus is designed to be lightweight for easy portability and use in public facilities. The exterior of the spray dispenser is formed from two pieces openable to form a housing. The housing contains an inner chamber for the aerosol mechanism. The aerosol mechanism comprises aerosol canisters and a triggering device. The canisters preferably contain different compounds. The triggering device, comprising a plunger and standard aerosol valve, is attached to a three-way switch for activating the canister and dispensing the spray directionally through a nozzle.

This application claims the priority of US Provisional Application Ser. No. 60/252,362 filed on Nov. 21, 2000.

### FIELD OF THE INVENTION

The present invention is in the field of dispensers. More specifically, the present invention relates to an aerosol dispenser that contains a plurality of aerosol cartridges in a container that emits a directed spray from a selected cartridge through one a preselected spray chamber.

### BACKGROUND OF THE INVENTION

Even after the horrible event of Sep. 11, 2001, individuals continue to use public facilities. People still attend sporting and cultural events. More people travel on inter-city busses and trains. People who travel by air may spend endless hours in airports. Inevitably such people are requited to use shared and public sanitary facilities.

In many public areas, a person is confronted with the risk of bacterial exposure through contact with commonly shared fixtures. For example, public restrooms potentially teem with countless bacteria on doorknobs, faucets, toilets, towel dispensers and other fixtures. Of necessity, a user must touch these fixtures in order to obtain the benefit thereof

In particular, given the increased public awareness of possible biological contamination particularly in such facilities, a prudent individual could well desire to sanitize such facilities prior to using them. In addition a person might desire to treat there hands, either before or after use, or spray a pleasant scent into the air either before, during, or after such use. Such ability would be enhanced if it could be accomplished while handling only a single container. Applicant believes that this desire presently cannot be met. Because no portable, personal, selectable, sanitizing spray container presently exists. Meeting this need is the primary purpose of the present invention.

### BRIEF DESCRIPTION OF THE INVENTION

The present invention is a personally portable aerosol spray device containing multiple aerosol canisters at least one of which is likely a cleaning or sanitizing agent. Using the present device, an agent, for example an antibacterial agent, can be easily sprayed on any surface to help protect against exposure to bacteria and reduce the potential of contracting a resultant infection. Other canisters within the container might contain cleaners, scent, or other desired composition. The device is small and lightweight, can be carried in a pocket or purse and be readily available for easy use in public places. In a simple embodiment, the device has housing made up of two similar tubular sections. Hereafter the discussion of the present invention will presume the two-chamber embodiment. Each tubular section is closed at one end and open at the other.

The open ends can be reversibly joined together in coaxial alignment and provide a housing with an interior space and a closed ends. The joint in the housing formed by the open ends of the tubular section allows the housing to be dissembled to access the interior space to replace used aerosol canisters when desired. In an alternative embodiment, the joint would not be readily dissembled and when the aerosol is expended, the device would be discarded.

The closed end of each tubular section has an aerosol discharge path incorporated into it. The discharge path is in concentric alignment with the axial bore of the tubular section. A trigger mechanism is incorporated into the housing. The trigger mechanism comprises a switch slidably disposed proximate the joint on an exterior surface of the housing. The switch has a normal middle position and a first and second position when displaced axially along the exterior surface of the housing. The switch is in direct mechanical communication with the plunger disposed in the interior space of the housing. The plunger of the trigger mechanism substantially divides the interior space of the housing into two similar chambers.

Each chamber can house an aerosol canister. Small aerosol canisters for dispensing different compositions as may be practiced in the present invention. For example one chamber might contain a sanitizing agent, and the other chamber might contain a beauty-enhancing product. The canisters are removable and replaceable if the joint in the housing can be dissembled. Each canister has a bottom that is in mechanical communication with the plunger of the trigger mechanism. The nozzle of each canister is in flow communication with the discharge path in the closed end of the tubular section.

Accordingly a prime object of the present invention is to provide a portable multiple spray dispenser container having a plurality of operable aerosol canisters housed therewithin and operative therefrom.

Another object of the present invention is to provide a portable container having a plurality of aerosol canister containing a plurality of compositions.

A further object of the present invention is to provide a container for a plurality of aerosol canisters where the canisters can selectively spray through an opening in the container.

These and still further objects as shall hereinafter appear are fulfilled by the present invention in a remarkably unexpected manner as can readily be discerned from the following detailed description of an exemplary embodiment of the present invention particularly when read in conjunction with the accompanying drawings in which like parts bear like reference throughout the several views.

### BRIEF DESCRIPTION OF THE DRAWINGS

FIG. 1 is a perspective view showing the multiple spray dispenser.

FIG. 2A is a perspective view of the spray dispenser showing the switch in one on position.

FIG. 2B is a perspective view of the spray dispenser showing the switch in the alternate position.

FIGS. 3A and 3B are perspective views of the two tubular sections of the housing showing the complementary threaded open ends and the relationship of the trigger mechanism to the housing.

FIG. 4 is a cross sectional view of the spray dispenser showing the aerosol canisters placed in the interior of the housing.

FIG. 5 is a cross-section of a closed end of the tubular sections of the housing showing the discharge path and how a canister is received and engaged in the section.

### DETAILED DESCRIPTION OF THE PREFERRED EMBODIMENT

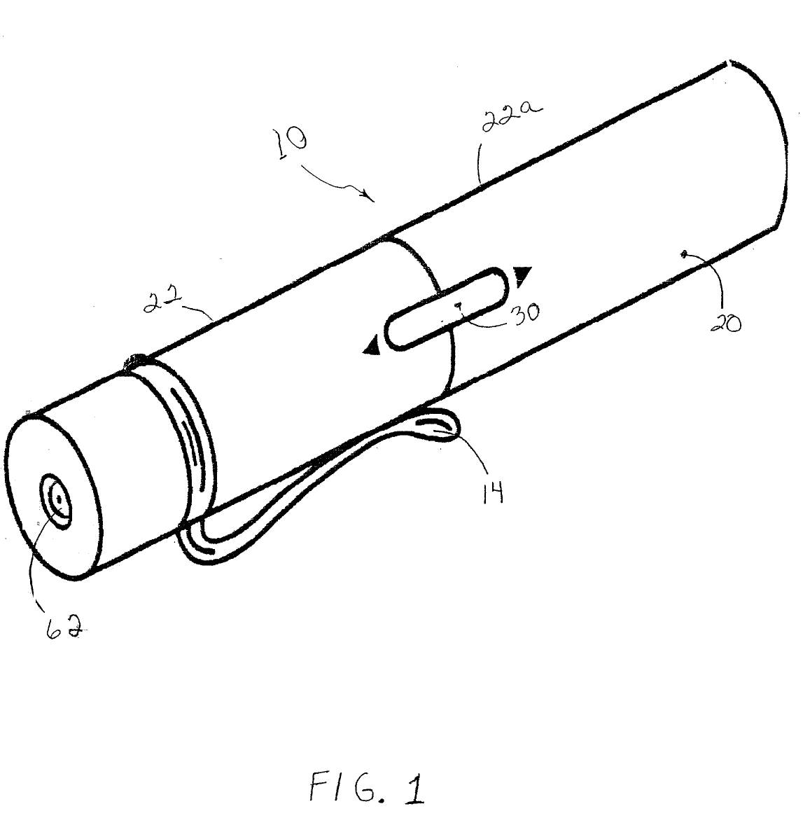

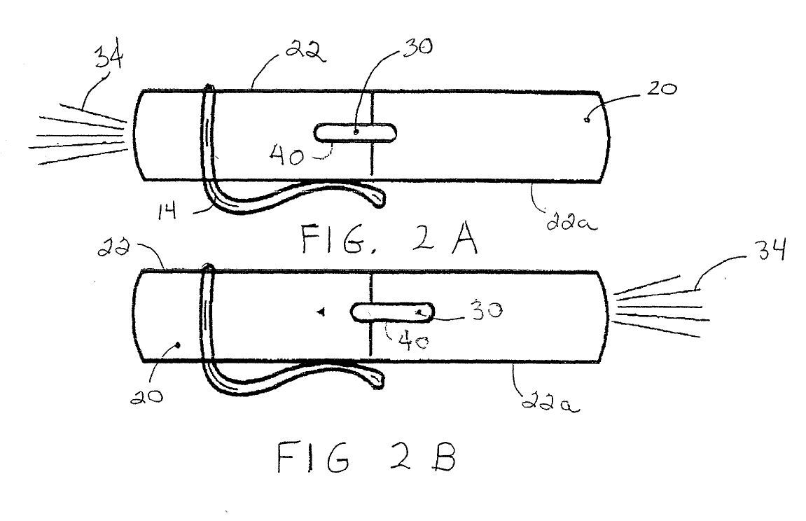

As shown in FIG. 1, the multiple spray device 10 of the present invention comprises a housing 20 and an aerosol trigger mechanism 30. In the preferred embodiment illustrated, the housing is substantially cylindrical, and contains two aerosol canisters. Hereafter the description of the preferred embodiment of the present invention shall be based upon this embodiment. As shown in FIGS. 2A and 2B sliding the trigger 30 in either direction from its normally neutral position causes an aerosol spray to be emitted from the device 10 in the direction in which the switch is slid. The housing 20 has two tubular sections 22 & 22a of symmetrical shape which can be opened to access the interior space 26, which contains two aerosol canisters 44, and closed again.

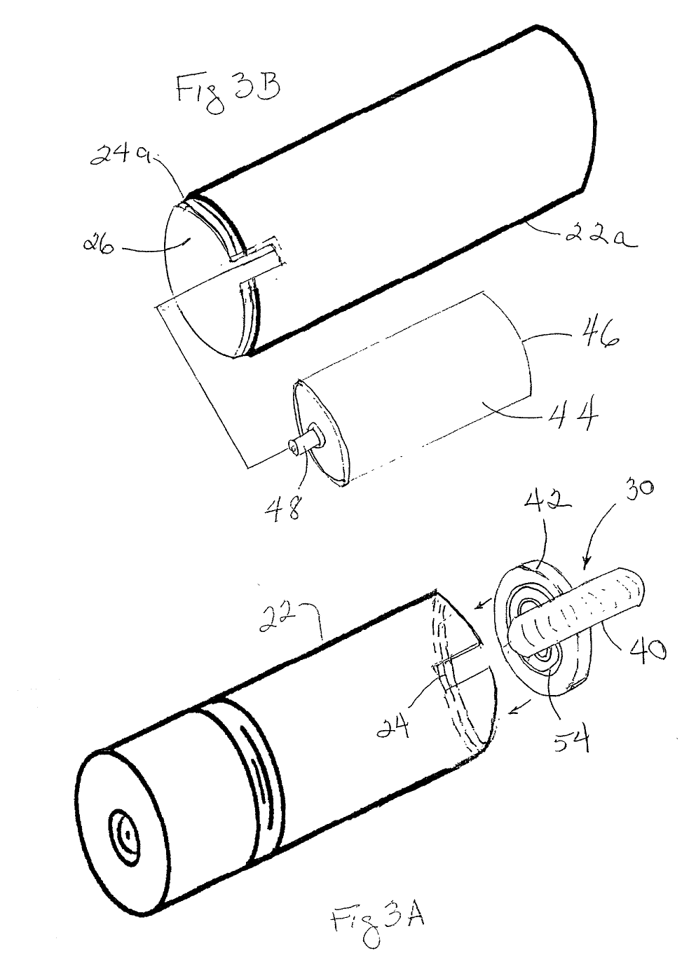

As shown in FIG. 3, the sections 22 & 22a have complementary threaded open ends 24 & 24a and are joined by screwing the complementary threaded ends open ends 24 & 24a together. The threaded open end 24 of one tubular section 22 mates with the complementary threaded open end 24a of the other tubular section 22a and screws together to form the housing 20 with an interior compartment 26. Alternatively, the two sections 22 & 22a may be joined by means of a snap or detent interface (not shown) at the open ends 24 & 24a the two tubular sections 22 & 22a. Means of connecting two tubular sections are known in the art and are readily practicable in the present invention by one of ordinary skill in the art.

The housing 20 may be made from various materials such as plastic or a lightweight metal allowing for easy portability of the antibacterial spray device 10. The housing could alternatively be made from a heavy cardboard stock in the case of disposable embodiment. The housing 20 also has a clip 14 attached to one of the tubular section 22 & 22a for securing the spray device 10 to a purse or pocket.

The housing of the present device 10 is closed at each end. Each closed end has a discharge path incorporated into it. Further, the discharge path comprises a nozzle seat 60 for receiving the nozzle of an aerosol canister 44, an aerosol passage that terminates in a discharge port 62. The aerosol passage is in flow communication with the nozzle seat 60 and hence with the nozzle 48 of the canister 44. When aerosol is discharged from the canister 44, it passes from the nozzle into the aerosol passage and is emitted from the discharge port 62 in a spray pattern determined by the configuration of the port 62 itself.

The chambers 56 inside the housing 20 each have a length selected to allow the trigger mechanism 30 to normally hold the nozzle 48 of each canister 44 in non-operative engagement with a nozzle seat 60. The switch 40 of the trigger mechanism 30 is slideable from its normal position a distance along the axis of the housing 20 in either direction sufficient to cause compression of the bottom 46 of the canister 44 and its nozzle 48 against the nozzle seat 60 to activate the discharge of the content of the canister 44. Optionally, the plunger 42 of the trigger mechanism 30 may include a normally fully compressed biasing spring 54. The spring 54 is fully compressed against the plunger 42 by the bottom 46 of the canister 44 in non-operative engagement with the nozzle seat 60 when the switch 40 of the trigger mechanism 30 is slid to toward the other canister 44.

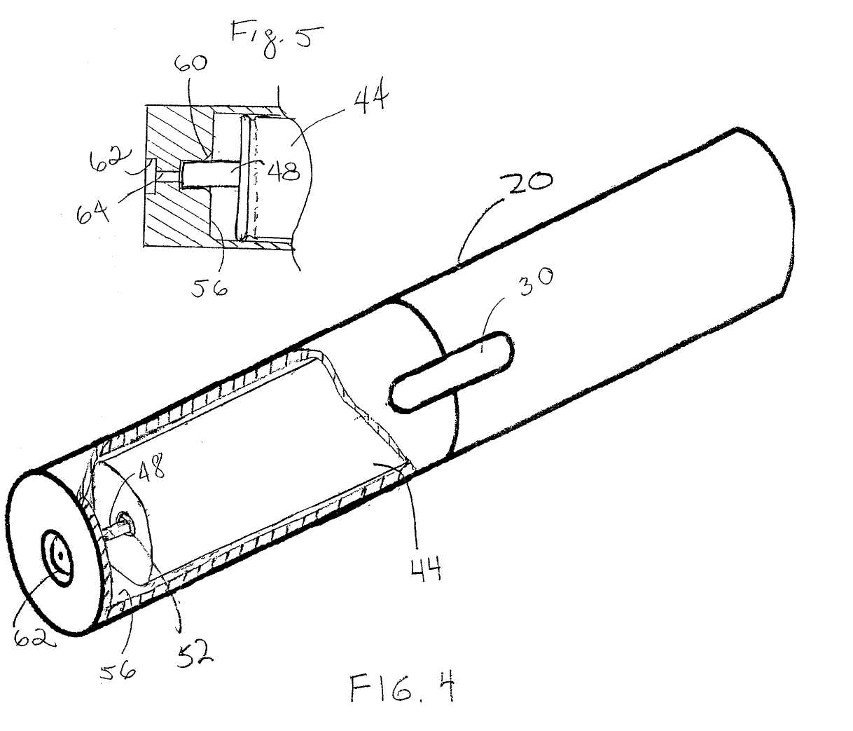

In the preferred embodiment, as shown in FIG. 4, dual aerosol cartridges 44 are contained the chamber 56 of the interior compartment 26 of the housing 20. Each cartridge 44 connects to the discharge port 62 by a flow passage 64 communicating with the nozzle 48 extending from the aerosol cadge 44. The aerosol catridge's 44 contents are pressurized. The trigger mechanism 30 includes a sliding switch 40.

To emit a spray 34, the switch 40 is moved to the left or right from the normally central off position. When the switch 40 is moved, the plunger 42 triggering mechanism 30 applies force to the bottom of the aerosol canister 44. Nozzle seat 60 prevents movement of the direction of the force applied by the plunger, causing compression of the canister nozzle 48 into the canister 44 activates the discharge of the pressurized content of the canister 44 through the nozzle 46.

When the pressurized contents of the aerosol cartridge 44 are released, they traverse flow passage and exit through the discharge port 62 as a spray 34. When the switch 40 is moved back to the off position, the force compressing the nozzle 48 into the canister 44 is removed and the nozzle 48 is expelled from the canister 44 by the intern bias and the spray 34 is shut off.

The switch 40 may either be of a manually operated type, where the switch is held in the position for spraying, or the switch 40 may have an automatic lock, remaining in the on position until manually switched off. Various other aerosol mechanisms are known in the art and are readily adaptable to the present invention by one having ordinary skill in the art.

While the above description contains many specifics, these should not be construed as limitations on the scope of the invention, but rather as exemplifications of one or another preferred embodiment thereof, Many other variations are possible, which would be obvious to one skilled in the art. Accordingly, the scope of the invention should be determined by the scope of the appended claims and their equivalents and not just by the embodiments.

The following is claimed

1. An multiple spray apparatus for portable use comprising:
a housing, comprised of symmetrically shaped pieces, each of said pieces having a spray directing exit, that form a chamber therewithin when assemble; and
an aerosol canister disposed within said chamber so that said canister may be selectively actuated to deliver a directed spray through said housing.

2. The apparatus according to claim 1 wherein said exit comprises a nozzle

3. The apparatus according to claim 1, wherein a securing clip is attached to said housing.

4. The apparatus of claim 1, wherein said aerosol canisters contain different compositions.

5. The apparatus of claim 1, also comprising a triggering mechanism whereby said aerosol canister is activated by a plunger operatively coacting with a standard aerosol valve.

6. The apparatus according to claim 1, wherein said plunger is connected to said nozzle to control the dispensing of said spray.

7. The apparatus according to claim 1, wherein said triggering mechanism is operatively connected to a three-way switch.

8. A personally portable aerosol spray device comprising:
a housing having two similar tubular sections, each said tubular section being closed at one end and open at the other end, and reversibly joined together at their said open ends in coaxial alignment and forming an interior space inside said housing and a closed end at each end of said housing;
a means for joining said open ends of said tubular sections together in coaxial alignment to form a joint between said sections;
a trigger mechanism slideably disposed proximate said joint on an exterior surface of said housing and in mechanical communication with the interior space of said housing, situate so that said trigger mechanism divides said interior space into two similar chambers; and
an aerosol canister removably housed in each said chamber, said canister having a bottom in mechanical communication with said trigger mechanism and said nozzle in flow communication with said discharge path.

9. The personally portable aerosol spray device of claim 1, wherein said housing has two similar tubular sections that are substantially cylindrical.

10. The personally portable aerosol spray device of claim 1, wherein the means for joining the said open ends of said tubular sections together in coaxial alignment further comprises said open ends each having a threaded component complementary to the other for screwing said sections together to form a joint.

11. The personally portable aerosol spray device of claim 1, wherein the closed end of each tubular section has a discharge path further comprising a nozzle seat for receiving the nozzle of the canister, an aerosol passage terminating in a discharge port, the aerosol passage being in flow communication with said nozzle seat.

12. The personally portable aerosol spray device of claim 1, wherein said chambers each have a length disposed to have said trigger mechanism normally hold the nozzle of each canister in non-operative engagement with a nozzle seat.

13. The device of claim 12, wherein said trigger mechanism is slideable from its normal position a distance along the axis of said housing in either direction sufficient to cause compression of the bottom of said canister and said nozzle against the nozzle seat to activate the discharge of the contents of said canister.

14. The device of claim 12, wherein said trigger mechanism further comprises a normally fully compressed biasing spring in mechanical communication with the bottom of each said canister, said spring to provide sufficient bias to hold said nozzle of said canister in non-operative engagement with said nozzle seat when said trigger mechanism is slid to toward the other said canister.
